SEQUENCE LISTING (6 sequences)

SEQ ID NO: 1 - DNA, 20 bp, Artificial Sequence
  Synthetic forward primer of MMP-1
gatgtggagtgcctgatgtg

SEQ ID NO: 2 - DNA, 20 bp, Artificial Sequence
  Synthetic reverse primer of MMP-1
tgcttgaccctcagagacct

SEQ ID NO: 3 - DNA, 20 bp, Artificial Sequence
  Synthetic forward primer of MMP-13
ggagcctctcagtcatggag

SEQ ID NO: 4 - DNA, 20 bp, Artificial Sequence
  Synthetic reverse primer of MMP-13
ttgagctggactcattgtcg

SEQ ID NO: 5 - DNA, 20 bp, Artificial Sequence
  Synthetic forward primer of GAPDH
gagtcaacggatttggtcgt

SEQ ID NO: 6 - DNA, 20 bp, Artificial Sequence
  Synthetic reverse primer of GAPDH
gacaagcttcccgttctcag

(19) United States
(12) Patent Application Publication
(10) Pub. No.: US 2013/0345168 A1
(43) Pub. Date: Dec. 26, 2013

(54) COSMETIC COMPOSITION FOR PREVENTING SKIN AGING CONTAINING CHITOOLIGOSACCHARIDES

(71) Applicants: Se-Kwon Kim, Dongnae-gu (KR); Chang-Suk Kong, Yeonje-gu (KR); Soon-Sun Bak, Seogu (KR); Jung-Ae Kim, Nam-gu (KR); Byul-Nim Ahn, Busanjin-gu (KR)

(72) Inventors: Se-Kwon Kim, Dongnae-gu (KR); Chang-Suk Kong, Yeonje-gu (KR); Soon-Sun Bak, Seogu (KR); Jung-Ae Kim, Nam-gu (KR); Byul-Nim Ahn, Busanjin-gu (KR)

(73) Assignee: PUKYONG NATIONAL UNIVERSITY INDUSTRY UNIVERSITY COOPERATION FOUNDATION, BUSAN (KR)

(21) Appl. No.: 13/885,302

(22) PCT Filed: Apr. 29, 2011
(86) PCT No.: PCT/KR11/03195

§ 371 Date: Jul. 31, 2013

(30) Foreign Application Priority Data
Nov. 15, 2010 (KR) 10-2010-0113572

(51) Int. Cl. A61K 8/73; A61Q 19/08

(52) U.S. Cl. CPC A61K 8/736; A61Q 19/08
USPC 514/55; 536/20

(57) ABSTRACT

The present invention relates to a composition for preventing skin aging containing chitooligosaccharides as an active ingredient. The invention provides a cosmetic composition for preventing skin aging caused by UV rays containing chitooligosaccharides of 1-3 kDa, 3-5 kDa or 5-10 kDa molecular weight as an active ingredient.

### FIELD OF THE INVENTION

The present invention relates to a composition for preventing skin aging containing chitooligosaccharides as an active ingredient. In particular, the invention relates to a cosmetic composition for preventing human skin aging caused by UV rays containing chitooligosaccharides less than 10kDa of molecular weight, as an active ingredient.

### BACKGROUND OF THE INVENTION

The functions of all organs in the human body gradually diminish with aging. The skin internally ages as well as all other organs do(intrinsic aging). Besides, the skin ages due to exposure to different harmful environmental condition(extrinsic aging). In particular, ultraviolet(UV) irradiation from sunlight is major environmental factor, stimulating aging of human skin. There are two types of aging for the skin based on these factors; intrinsic aging and photoaging. The signs of aging are: wrinkle formation, loss of skin tone and sagging skin etc. These are observed from photoaging induced by UV irradiation as well as intrinsic aging. While natural aged skin is characterized by thin, smooth and soft wrinkles, photoaged skin refers to thick and coarse wrinkles. Thus, it is most important for keeping young skin to protect the skin against exposure to the sun.

The three types of UV radiation are classified according to their wavelength: UVA(400-315 nm), UVB(315-280 nm) and UVC(280-100 nm). Of these, UVB range has a region with strongest energy intensity. When overexposed to UVB, it plays a part in contributing to wrinkle formation as well as causing skin cancer. Molecular response of skin against UV exposure can be triggered by photochemical formation of reactive oxygen species(ROS). UV-induced ROS directly cause chemical oxidation of the cell components, such as lipid, protein and DNA. Besides, ROS caused by UV irradiation allow to increase the secretion of collagenolytic MMPs(metrix metalloproteinases) in human dermal fibroblasts, such as MMP-1(interstitial collagenase) and MMP-3(collagenase 3), that may damage natural fibrillar collagen. Thus, increases in the level of collagenolytic MMPs in quantities are responsible for wrinkle formation and reduction of skin elasticity.

Chitin and Chitosan are natural cationoid polysaccharides, which are found in crustacean shells, insect cuticle and cell walls of some microbes. Chitosan is made by treating chitin with the alkali deacetylation, which is composed of β(1,4)-linked N-acetyl-D-glucosamine unit, having multiple biological activities. Chitooligosaccharide(COS) is hydrolyzed to chitosan derivatives. Unlike chitosan, COS has shorter chain length and free amino radical in D-glucosamine unit.

In preferred comparison among chitin, chitosan and chitooligosaccharide involve the following:

Chitin, a polymer of a N-acetyl-D-glucosamine unit over 5,000, which of the molecular weight is over 1 million, i.e. more than 100kDa, and chitosan, a polymer of D-glucosamine unit over 5,000 removed acetyl group from chitin, which of the molecular weight is over 1 million, i.e. more than 100 kDa, in contrast, chitooligosaccharide is obtained by hydrolyzing to less than 10 of D-glucosamine unit of chitosan, resulted in less than 10 kDa of the molecular weight.

In addition, chitin and chitosan are polymers formed by repeating units of sugar, so that could have many hydroxyl groups inside the molecule, thus noted for its water holding capacity. It is used for the moisturizer after the procedure of N-acylation due to being insoluble in water but soluble in weak acid(e.g. about 1% acetic acid). In contrast, chitooligosaccharide is soluble in water as well as has capability to hold water so that could have many hydroxyl groups in the molecule.

For these reasons, chitooligosaccharide is soluble in natural solutions, thus adapted to in vivo and in vitro systems. Chitooligosaccharide is noted for antitumor, antifungal, antibacterial, antiviral and ROS scavenging effect depending on its range of the molecular weight.

With regard to prior art associated with chitooligosaccharide, there are a large number of arts on food containing chitooligosaccharide. KR 10-2008-0049175 describes compositions containing chitooligosaccharide for recovering fatigue, which affect AMPK and enzymes related to fat metabolism, the resulting accelerate energy metabolism in hepatocyte. KR 10-2010-0062137 describes the pharmaceutical compositions with anti-oxidative activity, containing aminoethyl chitooligosaccharide as active ingredient, COS derivative having anti-aging activity. However studies are still needed for inhibitory effect of chitooligosaccharide on skin aging.

### DETAILED DESCRIPTION OF THE INVENTION

### Technical Field

The invention is based on the finding with mentioned above, which purposes to provide a composition for preventing skin aging containing chitooligosaccharide as an active ingredient.

### Technical Solution

The purpose of the invention could be achieved by treating with chitooligosaccharide to Human dermal fibroblasts that is exposed to UV, then proceeding to determine and analyze cytotoxic effects, ROS scavening activity, protective effects on oxidative DNA damage, inhibitory effects on MMP expression and inhibitory effects on collagen degradation, identifying the inhibitory effects on skin aging.

### EFFECTS OF THE INVENTION

The invention has an outstanding effect on disclosing anti-aging effects of chitooligosaccharide and providing a composition having the efficacy on anti-aging.

### BRIEF DESCRIPTION OF THE DRAWINGS

FIG. 1 shows the cytotoxicity of Human dermal fibroblasts exposed to different intensity of UVB irradiation. After cells were exposed to 50-200 mJ/cm² of UVB irradiation, the cytotoxicity was measured by (A) MTT assay and (B) LDH release assay. Means with different letters are significantly different by Duncan's multiple range test(p<0.05). Blank: not exposed to UVB.

FIG. 2 shows the effects of different molecular weights of COSs on cell viability of Human Dermal Fibroblasts exposed to 100 mJ/cm² of UVB irradation. After cells were exposed to 100 mJ/cm² of UVB irradiation, it were treated with different molecular weights(1-3 kDa, 3-5 kDa, 5-10 kDa) of COS. The cell viability was measured by (A)MTT assay and (B)LDH release assay. Blank: not exposed to UVB, control: only exposed to UVB.

FIG. 3 shows the effects of different molecular weights of COSs on cellular ROS synthesis induced by UVB irradation. Cells exposed to 100 mJ/cm² of UVB irradiation were treated with COS, then incubated for 48 hour and filled with DCFH-DA (Dichlorofluorescein diacetate). After staining with DCFH-DA, ROS was detected using fluorescence spectrophotometry.

FIG. 4 shows the effects of different molecular weights of COSs in UVB-induced DNA oxidative damage. After the cells were exposed to 100 mJ/cm² of UVB irradiation, then it were treated with different molecular weights of COSs. DNA was isolated from UVB-induced cell with or without treating with COS, followed by running on a 1% agarose gel electrophoresis.

FIG. 5 shows the effects of different molecular weights of COSs on MMP expression in UVB-exposed Human Dermal Fibroblasts.

FIG. 6 shows the effects of COS(3-5 kDa) on collagen degradation(A), MAPK activation(B) and AP-1 activation(C) in UVB-exposed Human Dermal Fibroblasts.

### DESCRIPTION OF THE PREFERRED EMBODIMENT

The invention provides a cosmetic composition for preventing skin aging caused by UV rays containing chitooligosaccharide(COS) as an active ingredient.

Cosmetic compositions of the present invention for preventing skin aging include chitooligosaccharide, in the range of 0.05 to 8% by weight, preferably in the range of 1 to 2% by weight of the composition.

Cosmetic compositions of the invention for preventing skin aging may include the ingredients commonly used in cosmetic compositions, such as antioxidants, stabilizers, solubilizing agents, vitamins, conventional adjuvants, such as pigments and fragrances, and carriers.

The composition may be provided in a variety of products in fields such as, but not limited to, solutions, suspensions, emulsions, pastes, gels, creams, lotions, soaps, surfactant-bearing cleansing, oils, powder foundation, emulsion foundation, wax foundation and spray foundation. In further embodiments, the compositions may be formed such as toners, astringents, lotions, nourishing creams, massage creams, essences, eye creams, cleansing creams, cleansing forms, cleansing waters, packs, sprays or powders.

In certain embodiments, the compositions are provided in the form of pastes, creams or gels, as carriers being used animal oil, vegetable oil, wax, paraffin, starch, tragacanth, cellulose derivative, polyethylene glycol, silicon, bentonite, silica, talc and zinc oxide.

In certain embodiments, the compositions are provided in the form of powder or spray, as carriers comprising lactose, talc, silica, aluminum hydroxide, calcium silicate or polyamide powder, in the form of spray, optionally comprising propellant, such as chlorofluorohydrocarbon, propane/butane or dimethylether.

In certain embodiments, the compositions are provided in the form of solution or emulsion, as carriers comprising solvent, solubilizer or emulsifier.

In certain embodiments, the compositions are provided in the form of suspension, as carriers comprising liquid diluent , such as water, ethanol or propylene glycol, suspension such polyoxyethylene sorbitol ester, crystallite cellulose.

In certain embodiments, the compositions are provided in the form of surfactant-bearing cleansing, as carriers comprising aliphatic alcohol sulfate, aliphatic alcohol ether sulfate.

Additional aspects and details of the invention will be made evident from following examples. However, the examples in the specification is illustrative only, and in no way limits the scope and meaning of the invention or any exemplified form.

Chitooligosaccharide(COS) of different molecular weights(1-3 kDa, 3-5 kDa, 5-10 kDa) was purchased in Kitto Life Co.(Seoul, Korea). COS hydrolyzed chitosan by means of enzyme reaction in reactor system and filteration passing through the UF(ultra-filteration) membrane having MWCO of 1-3 kDa, 3-5 kDa and 5-10 kDa. COS of different molecular weights were dissolved in water for the purpose of cell culture.

Data were expressed as mean±SD. Statistical analysis was performed by the one way ANOVA of Statistical Analysis System(SAS v9.1, SAS Institute Inc., Cary, N.C., USA). A significant difference in the mean value between treatment means were determined using Duncan's multiple range tests(p<0.05).

### EXAMPLE 1

### Cell Culture

Human dermal fibroblasts(Modern cell & Tissue Technologies INC) were incubated in DMEM medium(Gibco-BRL, Gaithersburg, Md., USA) supplemented with 10% FBS, 2 mM glutamine and 100 μg/mL penicillin-streptomycin(Gibco-BRL, Gaithersburg, Md., USA) at 37° C. in a humidified, 5% CO₂ in air incubator.

### EXAMPLE 2

### UVB-Exposed Cell Viability and Effects of COS on Cytotoxicity

To determine appropriate energy levels on UVB irradiation, after above human dermal fibroblast cell in culture were exposed to different UVB sources in the range of 50-200 mJ/cm², measured cytotoxicity in comparison to data obtained by MTT assay and LDH release assay.

UVB Irradiation

To determine the optimal level of UVB irradiation intensity, the cells were seeded into 24-well plates containing DMEM supplemented with 10% FBS, 2 mM glutamine and 100 μg/mL penicillin-streptomycin at a density of 1×10⁵ cells/well, and incubated at 37° C. in a humidified 5% CO₂ in air. After 24 hour of incubation, the cells in each well of 200 μL PBS were exposed to UVB energy in the range of 50-200 mJ/cm²(312 nm UVB light source, Bio-Sun lamp, Vilber Lourmat, Marine, France). After irradiation, the cells were cultured in serum-free DMEM medium for 48 hours.

MTT Assay

The levels of HDF(human dermal fibroblast) cell viability were determined by means of mitochondrial activity which converts 3-[4,5-dimethylthiazol-2-yl]-2,5-diphenyl tetrazolium bromide(MTT) into its insoluble formazan products. The cells were cultured into 96-well plates at a density of 1×10⁴ cells/well. After 24 hours of incubation, the cells were stimulated by means of UVB irradiation(100 mJ/cm²) and then cultured at 37° C. in a humidified, 5% CO₂ in air for 48 hour with or without COS sample. The supertanant medium was removed and 100 μl 1 mg/mL MTT reagent was added into each well, then incubated for 4 hours. After removal of MTT without conversion, DMSO(dimethyl sulfoxide) was added and measured OD(optical density) at 540 nm using microplate reader(Tacan Austria GmbH, Salzburg, Austria), thus determined amounts of formazan in living cells. Relative cell viability(%) was expressed as a percentage relative to the untreated control cells.

LDH Assay

LDH Cytotoxicity Detection Kit on market was used to assess cell damage by quantifying Lactate Dedrogenase(LDH)-release. Cells were exposed according to protocol. The conditioned media of UVB-exposed cell was collected for LDH measurements. After adding matrix mix solution to supernatant medium at a ratio of 1:1, followed by culture at 37° C. for 30 min under light-shading conditions. After that, 1N HCl stop solution was added, then immediately quantified absorbance at 490 nm by using microplate reader(Tacan Austria GmbH, Salzburg, Austria). 0.1%(w/v) Triton X-100 was set as control, which is defined as 100% LDH release.

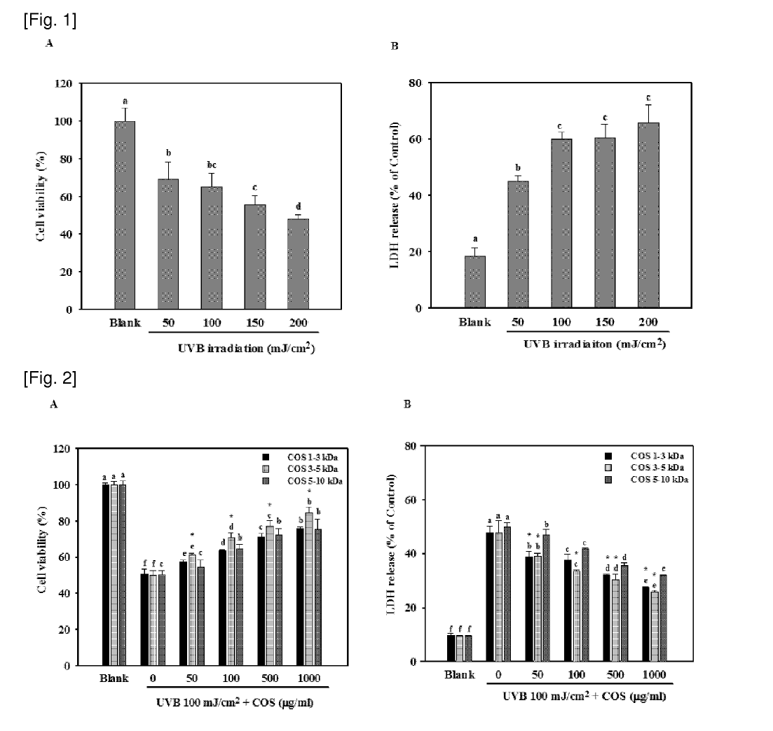

LDH is an enzyme found stable in many organs and cells, and LDH release from the cell into culture medium suggests that cells are breaking down. When the cells are exposed to UVB energy, cell viability has significantly decreased(FIG. 1A) and increased LDH release in a dose-dependent manner(FIG. 1B). Cells exposed to UVB energy over 100 mJ/cm² induce cytotoxicity in such a way similar to that of UVB-exposed cell.

Based on these results, we conducted an experiment on the effects of various molecular weight of COS at 100 mJ/cm² in the cell viability and the damage severity of UVB-exposed HDF cells. As the concentration of COS increased, cell viability virtually increased in dose-dependent manner relative to the only UVB-exposed cells(FIG. 2A). LDH release assay indicates that COS virtually reduces cell damage caused by UVB exposure in dose-dependent manner(FIG. 2B). Inhibitory effect on cell damage by UVB exposure is more effective in human fibroblast cell treated with COS(3-5 kDa) than other COSs.

### EXAMPLE 3

### Effects of COS on Intracellular ROS Scavening Activity in UVB-Exposed Cells

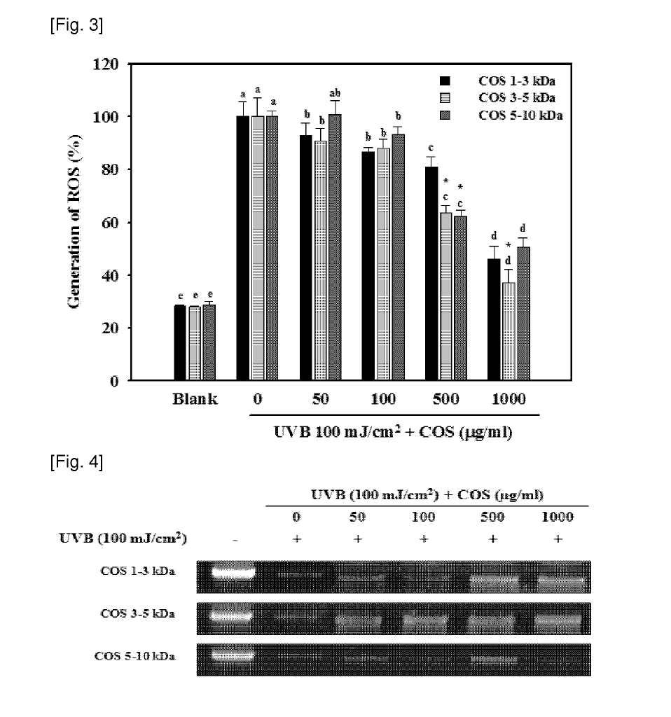

The intracellular ROS generation of cells was detected using the 2′,7′-dichlorofluorescin diacetate(DCFH-DA), the oxidative sensitive dye. After cultured in 96-well microplates for 24 hours, HDF cells were exposed to UVB(100 mJ/cm²). The exposed cells were treated with COS for 48 hours, and then filled with 20 μM DCFH-DA dissolved in PBS, and incubated for 30 minutes in the darkroom at 37° C., 5% CO₂. The cells finally were washed twice in PBS, then DCF fluorescence was detected by fluorescence microplate reader(Tacan Austria GmbH, Salzburg, Austria) with maximum excitation and emission spectra of 485 nm and 535 nm, respectively. The results are indicated in FIG. 3.
Increase of DCF fluorescence was observed depending on UVB exposure. UVB-exposed group indicate the strength of the fluorescence levels 3 times higher than the blank group that is not exposed to UVB. In the presence of COS, it significantly allowed to decrease the strength of the fluorescence on DCF in UVB-exposed cell in dose-dependent manner, which demonstrate increases in scavening activity on intracellular ROS production(p<0.05). Of COSs, 3-5 kDa COS effectively allows to inhibit UVB-mediated ROS production by means of UVB irradiation. 1-3 kDa COS and 5-10 kDa COS indicate the lower UVB-irradiated ROS scavening activities than 3-5 kDa COS.

### EXAMPLE 4

### Inhibitory Effects of COS on UVB-Induced DNA Damage

Genomic DNA was extracted from HDF cell by using the slightly modified standard phenol/protease K method. After washing UVB-irradiated cells twice with PBS, which was gathered by using 1 mL PBS comprising 10 mM EDTA. It were centrifuged at 13,400×g for 5 minutes at 4° C., then the resulting precipitated cells were resuspended in 410 μl solution comprising RNase A(0.5 mg/mL), protease K(10 mg/mL), SDS(10%) and NaOAC(0.2M). And the resulting mixture was cultured for 30 minutes at 37° C. and 1 hours at 55° C. After that, 1 volume of phenol:chloroform:isoamyl alcohol(25:24:1) was added to 1 volume of sample, then centrifuged at 13,400×g for 5 minutes at 4° C. Then, transferred the upper, aqueous phase to a fresh Effendorf tube, added 1.5 volume of 100% cold ethanol to 1 volume sample, incubated for 30 minutes at −20° C. After centrifugation at 5,900×g for 5 minutes at 4° C., the supertanant was removed and dissolve the remaining pellet with 20 μl TE buffer(10 mM Tris-HCl, 1 mM EDTA, pH 8.0). The ratio of the absorbance at 260 and 280 nm is used to assess the purity of DNA, using spectrophotometric measurements.

The reaction mixtures of aliquot(20 μl) containing 1 μg DNA were separated by 1% agarose gel electrophoresis at 100V for 10 min. The gels were stained by 1 mg/mL EtBr(ethidium bromide) for 30 min, then photographed under UV light using AlphaEase Gel Image Analysis Software(Alpha Innotech, San Leandro, Calif., USA). The results are indicated in FIG. 4.

DNA damage was observed in UVB-exposed cell, which was decreased in dose-dependent manner in the presence of COS. It shows that these results indicate appropriate protective effects on UVB-mediated DNA damage. In particular, 3-5 kDa COS effectively protects DNA damage due to UVB exposure.

### EXAMPLE 5

### RT-PCR(Reverse Transcriptase Polymerase Chain Reaction) Analysis

Total cellular RNA was isolated using Trizol reagent(Invitrogen Co., Calif., USA). 2 μg of RNA isolated was reverse transcribed into complementary DNA(cDNA) by using oligo(dT) primers(Promega, Madison, Wis., USA). The targeted cDNA was amplified by using the forward primer's sequence and the reverse primer's sequence shown positioned below: the MMP-1 forward primer is: 5′-GAT-GTG-GAG-TGG-CTG-ATG-TG-3′, and reverse primer is: 5′-TGC-TTG-ACC-CTC-AGA-GAC-CT-3′; the MMP-13 forward primer is: 5′-GGA-GCC-TCT-CAG-TCA-TGG-AG-3′, and reverse primer is: 5′-TTG-AGC-TGG-ACT-CAT-TGT-CG-3′; the GAPDH forward primer is: 5′-GAG-TCA-ACG-GAT-TTG-GTC-GT-3′, and reverse primer is: 5′-GAC-AAG-CTT-CCC-GTT-CTC-AG-3′. The resulting was repeatedly amplified for 35 cycles with 45 sec at 95° C., 50 sec at 60° C., 60 sec at 72° C. After amplification, the annealing step proceeded continuously at 72° C. for 5 minutes. PCR products were separated by electrophoresis in 1% agarose gels at 100V for 10 minutes. Gels were strained with 1 mg/mL EtBr(ethidium bromide) and then photographed under UV light by using AlphaEase® gel image analysis software(Alpha Innotech., San Leandro, Calif., USA). Finally, the LAS 3000® luminescent image analyzer(Fujifilm Life Science, Tokyo, Japan) was used to determine relative density of bands on agar gel.

### EXAMPLE 6

### Western Blot Analysis

A whole cell was lysed in RIPA buffer(Sigma-Aldrich Corp., St.Louis, USA). After centrifugation, the total protein content of cytolysates were determined by using Lowry method(BioRad Laboratories, Hercules, Calif.). After supernatant fractions containing the same amount of protein were subjected to electrophoresis on 10% or 12% SDS-PAGE gel, and then transferred into the nitrocellulose membrane(Amersham Pharmacia Biotech., England, UK), and blocked at least for 1 hour with 5% skimmed milk in TBS with 0.1% Tween 20(TBS-T), and hybridized with primary antibody such as MMP-1, MMP-13, type I procollagen, type I collagen, pERK, pJNK, pp38, c-Jun, c-fos and pp53(Santa Cruz Biotechnology Inc., Calif., USA). All of the primary monoclonal antibody were diluted 1:1000 in TBS-T. The combined antibody was detected by using Horseradish peroxidase conjugated secondary antibody, and immune responsive protein was detected by using chemiluminescence ECL detection kit(Amersham Pharmacia Biosciences, England, UK). Western blotting bands was visualized by means of LAS3000 luminescent image analyzer(Fujifilm Life Science, Tokyo, Japan).

### EXAMPLE 7

### Effects of COS on UVB-Mediated MMP Expression

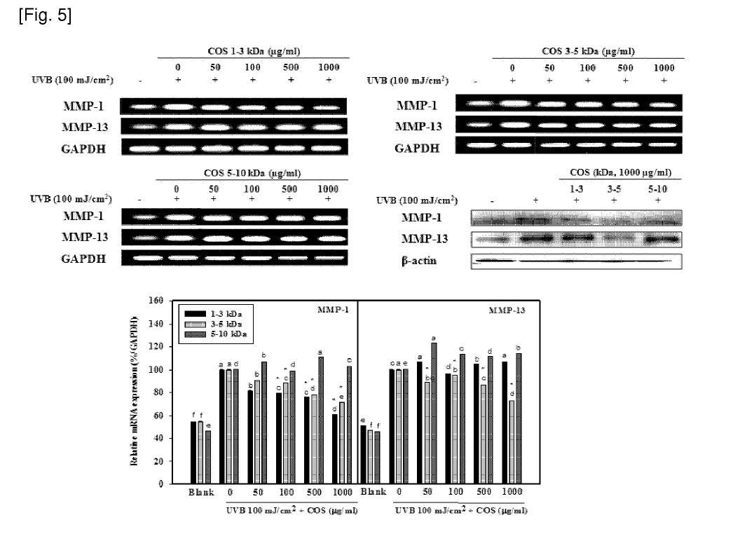

As described in Example 5 and 6, the effects on the expression of collagenolytic MMPs in UVB-exposed cells were determined using RT-PCR and Western blot analysis(Fig.5). The gene expression levels of MMP-1 and MMP-13 were significantly increased in only UVB-exposed cell. However, gene expression of UVB-mediated collagenolytic MMP in COS-treated, UVB-exposed was decreased. In particular, MMP-1 gene expression was significantly decreased by 1-3 kDa COS and 3-5 kDa COS in dose-dependent manner, also decreased by 3-5 kDa in dose-dependent manner.

According to these test data, 3-5 kDa COS among all COSs optimally showed the most protective effects on UVB-mediated photoaging.

### EXAMPLE 8

### Effects of COS(3-5 kDa) on Hydrolysis of Collagen in UVB-Exposed Cell

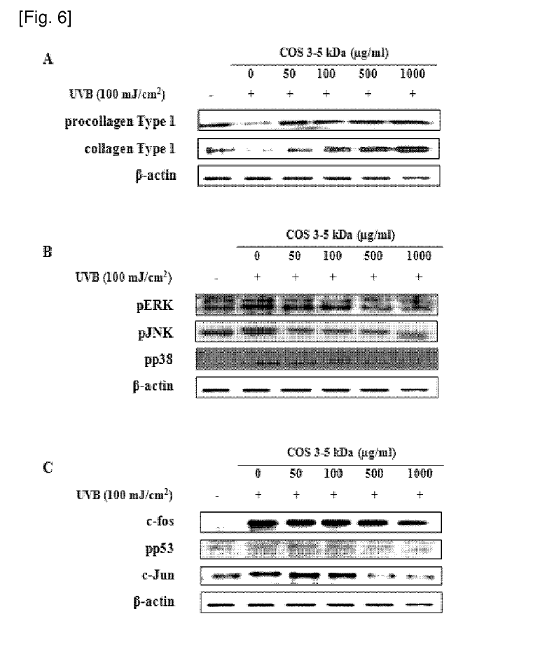

Experiments were conducted to examine the effects of COS on procollagen synthesis and collagen degradation in UVB-exposed cell, as described in Example 6.(FIG. 6A). Regulation of Intracellular type I procollagen and collagen protein resulted in decreasing in only UVB-exposed cell. The levels of type I procollagen and collagen in cell decreased by UVB exposure have increased in dose-dependent manner when 3-5 kDa COS was present.

### EXAMPLE 9

### Inhibitory Effects of 3-5 kDa COS in MAPK Pathway Activation

To determine signalling cascades reacting to the protective effects of COS in UVB-exposed cell, the experiments were performed for MAPK signalling pathway as described in Example 6(FIG. 6B). The effects of 3-5 kDa COS were investigated in the regulation of JNK(c-Jun N-terminal kinase), ERK1/2(extracellular signal-related kinase) and p38 MAPK, three major subgroups of MAPKs, in UVB-exposed cells. Phosphorylated JNK, p38 MAPK and ERK1/2 protein levels increased, in contrast the protein levels effectively decreased when treated with COS(3-5 kDa). AP-1(activator protein-1) transcription factor is a critical mediator of acute photodamage including the MMP expression and decreases of type I procollagen, which is a protein belonging to the Jun and Fos families. Thus, the effects of COS(3-5 kDa) in UVB-induced AP-1 activation were experimented. As a result, nuclear transcription factor c-Jun and c-fos activated by UVB irradiation was significantly weakened by treating with COS(3-5 kDa)(FIG. 6C). In addition, the phosphorylation of another transcription factor p53 was also inhibited by treating with COS(3-5 kDa).

As a result of above, we found that protective effect of COS on cytotoxicity of UVB-stressed human fibroblast relies on its molecular weight. COS suppress DNA damage and UVB irradiation-induced ROS(reactive oxygen species) production accompanied with down-regulation of MMP-1(Matrix metalloproteinase-1) and MMP-13. In a comparative analysis, COS(3-5 kDa) exhibit the most potent protective effect on UVB-stressed fibroblasts. What's more, the presence of COS(3-5 kDa) attenuates UVB-derived collagenolytic MMP production and collagen degradation. The photoprotective activation of COS(3-5 kDa) can be determined by transcriptional phosphorylation of MAPK(mitogen-activated protein kinase)-responsive signaling pathways.

1. A cosmetic composition for preventing skin aging comprising chitooligosaccharide as active ingredient.

2. The cosmetic composition for preventing skin aging of claim 1, wherein the chitooligosaccharide is selected from chitooligosaccharide with molecular weight 1-3 kDa, chitooligosaccharide with molecular weight 3-5 kDa and chitooligosaccharide with molecular weight 5-10 kDa.

3. The cosmetic composition for preventing skin aging of claim 1, wherein the molecular weight of chitooligosaccharide is 3-5 kDa.

4. A cosmetic composition for improving wrinkles of claim 1, wherein the chitooligosaccharide content is from 0.05% to 8% by weight on the basis of the total mass of the composition.

5. The cosmetic composition for preventing skin aging of claim 2, wherein the molecular weight of chitooligosaccharide is 3-5 kDa.
